(19) United States
(12) Patent Application Publication
(10) Pub. No.: US 2022/0128157 A1
(43) Pub. Date: Apr. 28, 2022

(54) Pinch Clamp for Flexible Tubing

(71) Applicants: Jason C. Nisler, Newton, NJ (US); Ralph M. Meola, Hackettstown, NJ (US); Ian Colquhoun, Frelinghuysen, NJ (US)

(72) Inventors: Jason C. Nisler, Newton, NJ (US); Ralph M. Meola, Hackettstown, NJ (US); Ian Colquhoun, Frelinghuysen, NJ (US)

(73) Assignee: BioFlex Solutions, LLC, Hackettstown, NJ (US)

(21) Appl. No.: 17/507,157

(22) Filed: Oct. 21, 2021

Related U.S. Application Data
(60) Provisional application No. 63/105,227, filed on Oct. 24, 2020.

(51) Int. Cl. F16K 7/06

(52) U.S. Cl. CPC F16K 7/061

(57) ABSTRACT

The pinch clamp has a locking assembly that includes two members for securing the jaw assemblies of the pinch clamp in place. The two members are a stud and a nut threaded on the stud. The stud has a spherical base pivotally mounted on an end of one jaw assembly and an arcuate boss projecting from the spherical base into mating sliding relation with the arcuate recess of the jaw assembly to limit pivoting of the base to a single plane. A threaded stern extends from the spherical base and is disposed in a bifurcation of the jaw assembly.

This is a Non-Provisional Patent Application end claims the benefit of Provisional Patent Application 63/105,227 filed Oct. 24, 2020.

This invention relates to a pinch clamp and more particularly to a pinch clamp for temporarily closing flexible tubing.

### BACKGROUND OF THE INVENTION

Pinching devices for flexible tubing are well known for use in the food and pharmaceutical industry for the temporary closure of fluid lines. Typically, pinching devices are used for specific sizes of tubing wherein most require the tubing to be inserted prior to connection.

U.S. Pat. No. 9,879,813 describes a pinching device that does not require the insertion of tubing through the device and that may be placed at various locations along fluid lines while actively in use. The pinching device is constructed with two members that pivot relative to each other to close and open on a flexible tubing with a locking nut assembly to lock the members in place.

### Objects of the Invention

It is an object of the invention to provide an improved pinch clamp for flexible tubing.

It is another object of the invention to provide a pinch clamp for flexible tubing that can be made of a minimum number of parts.

It is another object of the invention to simplify the locking together of a pair of pivotal members of a pinch clamp.

It is another object of the invention to simplify the manufacture of a pinch clamp for flexible tubing.

### BRIEF SUMMARY OF THE INVENTION

Briefly, the invention provides a pinch clamp for flexible tubing that comprises a pair of jaw assemblies that are pivotally connected to each other to close on a flexible tubing and a locking assembly for securing the jaw assemblies in place.

A first jaw assembly is of one-piece construction having a pinching bar, a pivot at one end of the pinching bar and an arcuate recess in an opposite end of the pinching bar.

A second jaw assembly is of one-piece construction having a pinching bar opposite the pinching bar of the first jaw assembly for pinching a flexible tubing therebetween, a hook portion at one end of pinching bar received on the pivot of the first jaw assembly, a bifurcation at an opposite end of the pinching bar and a recessed seat in an upper surface at the same end.

The locking assembly includes two members, namely, a stud and a nut threaded on the stud.

The stud has a spherical base pivotally mounted on an end of the first jaw assembly opposite the end containing the pivot, an arcuate boss projecting from the spherical base into mating sliding relation with the arcuate recess of the first jaw assembly to limit pivoting of the base to a single plane, and a threaded stem extending from the spherical base and disposed in the bifurcation of the first jaw assembly.

The nut is threaded on the threaded stern of the stud and is seated within the recessed seat of the second jaw assembly to secure the threaded stern in the second jaw assembly.

The pinch clamp is thus constructed of four members, i.e. two one-piece jaw assemblies, a stud and a nut. As such, the cost, of manufacture is minimized.

In addition, the first jaw assembly is formed with a part spherical seat for slidably receiving the spherical base of the stud and a resilient lip or protruding detents partially encompassing the spherical base opposite the seat to retain the spherical base within the part spherical seat. To this end, the stud dimension is larger than the lip on the jaw assembly receiving the stud to permit the spherical base to be snap-fitted into the first jaw assembly. Dimensional accuracy is critical to enable both parts to be manufactured from similar materials while ensuring that the parts can properly secure together. This construction allows the stud to be installed in the jaw assembly after manufacture without the need for a pivot pin such as with the pinching device of U.S. Pat. No. 9,879,813.

The nut is provided with a plurality of circumferentially spaced apart projections on the underside for selectively fitting into similarly spaced apart depressions in the recessed seat during rotation of the nut on the stem of the stud to lock the nut against loosening while preventing overtightening.

These and other objects and advantages of the invention will become more apparent from the following detailed description taken in conjunction with the accompanying drawings wherein:

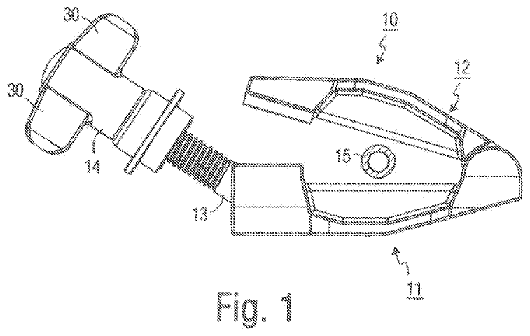

FIG. 1 illustrates a side view of pinch clamp in accordance with the invention in an opened condition;

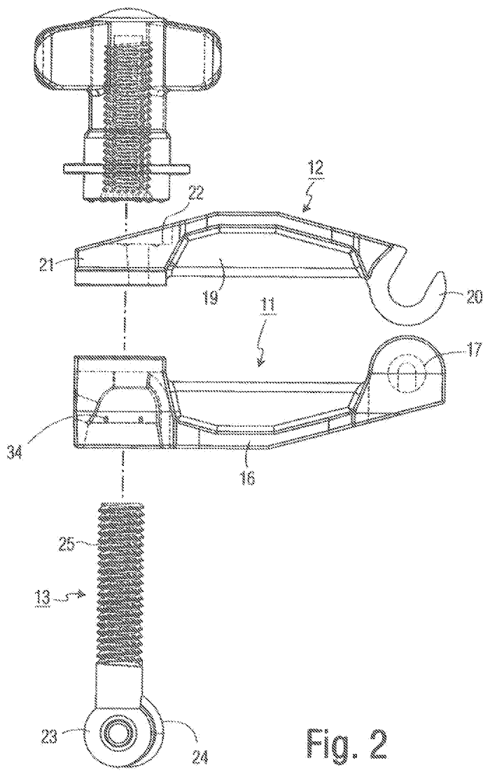

FIG. 2 illustrates an exploded view of the pinch clamp of FIG. 1;

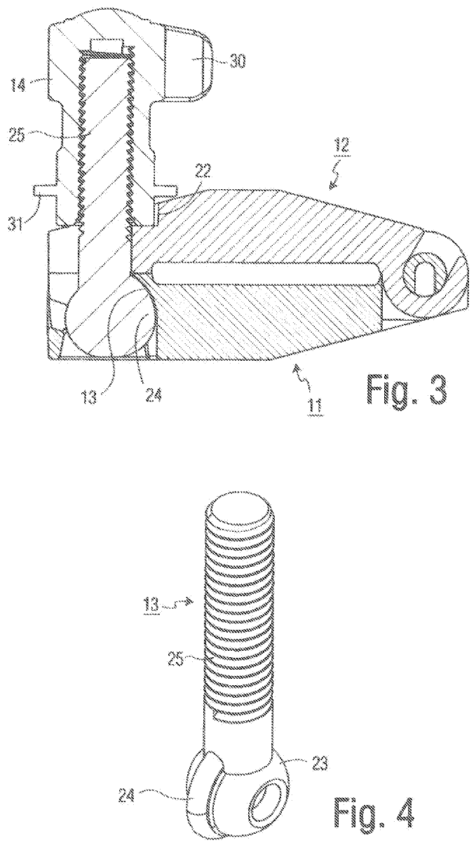

FIG. 3 illustrates a cross-sectional view of the pinch clamp of FIG. 1 in a closed condition;

FIG. 4 illustrates a perspective view of the stud of the pinch clamp of FIG. 1;

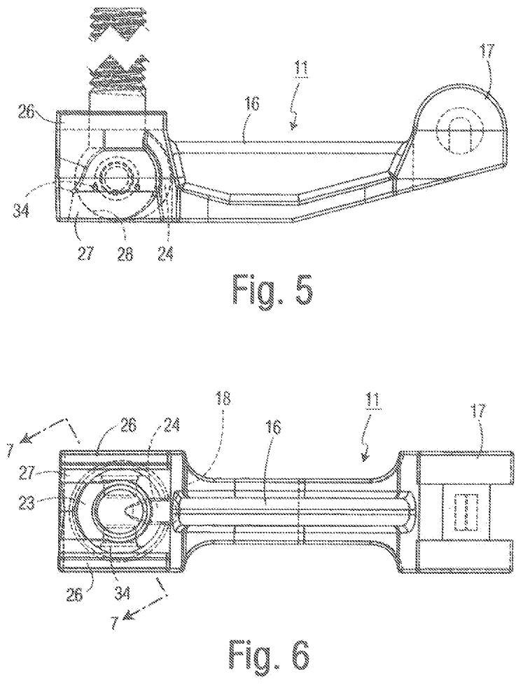

FIG. 5 illustrates a side view of the lower jaw assembly and stud of FIG. 2;

FIG. 6 illustrates a plan view of the lower jaw assembly of FIG. 2;

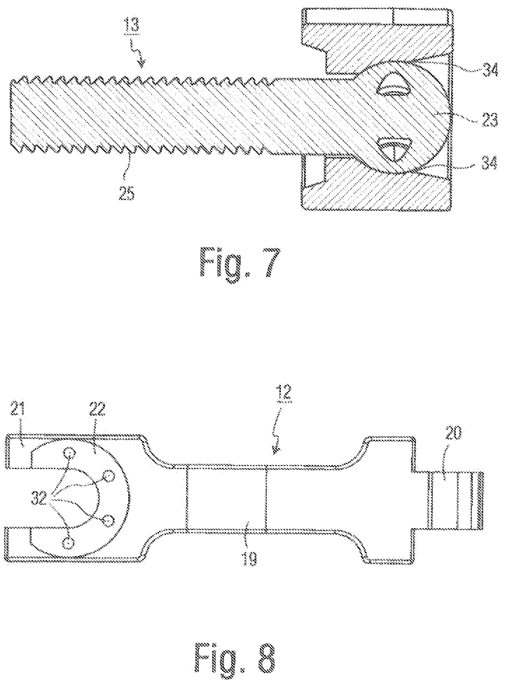

FIG. 7 illustrates a view taken on line 7-7 of FIG. 6; and

FIG. 8 illustrates a plan view of the upper jaw assembly of FIG. 1.

Referring to FIGS. 1 and 2, the pinch clamp 10 comprises two jaw assemblies 11, 12 and a locking assembly formed of a stud 13 and a nut 14 for a total of four separate members.

The pair of jaw assemblies 11, 12 are pivotally connected to each other to close on a flexible tubing 15 and the locking assembly 13, 14 serves to secure the jaw assemblies 11, 12 together in place while pinching the flexible tubing 15.

Typically, the jaw assemblies 11, 12 are made of a plastic material and are constructed to function in a manner' as described in U.S. Pat. No. 9,879,813.

Referring to FIGS. 1, 2, 5 and 6, the lower jaw assembly 11 as viewed, has a pinching bar 16, a pivot 17 at one end of the pinching bar 16 and an arcuate recess 18 (see FIG. 3) in an opposite end of the pinching bar 16.

Referring to FIGS. 1, 2 and 7, the second or upper jaw assembly 12, as viewed, has a pinching bar 19 opposite the pinching bar 16 of the lower jaw assembly 11 for pinching the flexible tubing 15 therebetween, a hook portion 20 at one end of the pinching bar 19 received on the pivot 17 of the lower jaw assembly 11, a bifurcation 21 at an opposite end of pinching bar 19 and a recessed seat 22 in an upper surface t said opposite end thereof.

Referring to FIGS. 2, 3 and 4, the stud 13 has a spherical base 23 pivotally mounted on an end of the pinching bar 16 of the lower jaw assembly 11, an arcuate boss 24 projecting from the spherical base 23 for mating sliding relation in and with the arcuate recess 18 in the end of the pinching bar 16 to limit pivoting of the base 23 to a single plane, and a threaded stem 25 extending from the spherical base 23 for disposition in the bifurcation 21 of the upper jaw assembly 12.

The pinching bar 16 of the lower jaw assembly 11 is formed at the end to receive the spherical base 23 of the stud 13 for pivoting relative to the lower jaw assembly in only one plane. To this end, as shown in FIGS. 5 and 6, the end of the pinching bar 16 has a part-spherical shape with the recess 18 therein, a pair of flat parallel side walls 26 extending therefrom and a floor 27 extending below and between the side walls 26 with a part-spherically shaped surface 28.

The part-spherically shaped end of the pinching bar 16 and floor 27 define a part spherical seat for slidably receiving the spherical base 23 of the stud 13.

When mounted in place, the spherical base 23 of the stud 13 is retained in the spherical seat by the non-concentric shape formed by the arcuate boss 24 and a resilient lip or protruding detents 34 while abutting the part-spherically shaped end of the pinching bar 16.

Referring to FIG. 3, the nut 14 is threaded on the threaded stem 25 of the stud 13 and has a pair of wings 30 for manually turning of the nut 14.

Referring to FIGS. 1, 2 and 3, the nut 14 includes a collar 31 that can be alternatively machined with slots or a hole by users. Upon location of the nut 14 within the recessed seat 22 of the upper jaw assembly 12 and tightening of the nut 14, the collar 31 can be secured against the upper jaw assembly 12 using a secondary tie (not shown) thereby holding the stud 13 in place to prevent the stud 13 and nut 14 threaded thereon from pivoting out of the bifurcated end of the upper jaw assembly 12.

Referring to FIG. 8, the recessed seat 22 of the upper jaw assembly 12 includes a plurality of spaced apart depressions 32 and the collar 31 on the nut 14 includes a plurality of spaced apart projections (not shown) for selectively fitting into the depressions 32 upon tightening of the collar 31 upon the recessed seat 22 thereby locking the nut 14 against loosening during use.

The pair of jaw assemblies 11, 12 are constructed in a manner as described in U.S. Pat. No. 9,879,813 such that the jaw assemblies 11, 12 interlock together to resist any side movement once seated in a closed position and are made of a resilient plastic material.

Referring to FIGS. 5, 6 and 7, in order to secure the stud 13 in the lower jaw assembly 11, the end of the pinching bar 16 is provided with a lip 34 that extends inwardly from the side walls 26 and part-spherically shaped end of the pinching bar 16 in order to partially encompass the spherical base 23 of the stud 13 from above, i.e. opposite the part spherical seat.

The stud 13 is made of a similar plastic to the plastic of the lower jaw assembly 11 and upon assembly of the pinch clamp 10, the spherical base 23 of the stud 13 is snap-fitted passed the lip 34 into the part spherical seat of the lower jaw assembly 11.

The invention thus provide a pinch clamp for flexible tubing that can be made of a minimum number of parts, i.e. four parts rather than six parts as is the pinching device described in U.S. Pat. No. 9,879,813, while simplifying the manufacture of the pinch clamp.

In addition, the invention provides a locking assembly for a pair of pivotally connected jaw assemblies of a pinch clamp that simplifies the locking together of the jaw assemblies.

What is claimed is:

1. A pinch clamp for a flexible tubing comprising a first jaw assembly having a first pinching bar a pivot at one end of said pinching bar and an arcuate recess in an opposite end of said pinching bar;
a second jaw assembly having a second pinching bar opposite said first pinching bar for pinching a flexible tubing therebetween, a hook portion at one end of said second pinching bar received on said pivot of said first jaw assembly, a bifurcation at an opposite end of said second rectilinear pinching bar and a recessed seat in an upper surface at said opposite end thereof;
a stud having a spherical base pivotally mounted on an end of said first jaw assembly opposite said one end of said first pinching bar, an arcuate boss projecting from said spherical base into mating sliding relation with said arcuate recess of said first jaw assembly to limit pivoting of said base to a single plane, and a threaded stem extending from said spherical base for selective disposition in said bifurcation of said first jaw assembly; and
a nut threaded on said threaded stem of said stud and seated within said recessed seat of said second jaw assembly to secure said threaded stem to said second jaw assembly upon disposition of said threaded stern in said bifurcation.

2. A pinch clamp as set forth in claim 1 wherein said first jaw assembly has a part spherical seat for slidably receiving said spherical base of said stud and a resilient lip partially encompassing said spherical base opposite said seat to retain said spherical base on said part spherical seat.

3. A pinch clamp as set forth in claim 2 wherein said stud is made of metal and said first jaw assembly is made of a resilient plastic material to permit said spherical base to be snap-fitted into said first jaw assembly.

4. A pinch clamp as set forth in claim 1 wherein said nut includes a collar for abutting on said recessed seat of said second jaw assembly.

5. A pinch clamp as set forth in claim 4 wherein said recessed seat of said second jaw assembly includes a plurality of spaced apart depressions and said collar includes a plurality of spaced apart projections for selectively fitting into said depressions upon disposition of said threaded stem in said bifurcation.

6. A pinch clamp for a flexible tubing comprising
a lower jaw assembly having a first pinching bar, a pivot at one end of said pinching bar and an arcuate recess in an opposite end of said pinching bar;
an upper jaw assembly having a second pinching bar opposite said first pinching bar for pinching a flexible tubing therebetween, a hook portion at one end of said second pinching bar received on said pivot of said first jaw assembly, a bifurcation at an opposite end of said second rectilinear pinching bar and a recessed seat in an upper surface at said opposite end thereof;
a stud having a spherical base pivotally mounted on an end of said lower jaw assembly opposite said one end of said first pinching bar, an arcuate boss projecting from said spherical base into mating sliding relation with said arcuate recess of said first jaw assembly to limit pivoting of said base to a single vertical plane, and a threaded stern extending from said spherical base and disposed in said bifurcation of said lower jaw assembly; and
a nut threaded on said threaded stem of said stud and seated within said recessed seat of said upper jaw assembly to secure said threaded stem to said upper jaw assembly.

7. A pinch clamp as set forth in claim 6 wherein said stud is made of metal and said lower jaw assembly is made of a resilient plastic material to permit said spherical base to be snap-fitted into said lower jaw assembly.
